(19) United States
(12) Patent Application Publication
(10) Pub. No.: US 2006/0120916 A1
(43) Pub. Date: Jun. 8, 2006

(54) Inhibiting biofilm formation by thermophilic microbes in paper and board machines

(75) Inventors: Marko Kolari, Vantaa (FI); Mirja Salkinoja-Salonen, Helsinki (FI); Hanna Laatikainen, Helsinki (FI); Paivi Tammela, Helsinki (FI); Pia Vuorela, Porvoo (FI); Pentti Vaatnen, Vaasa (FI); Terhi Johanna Hatunen, Vaasa (FI)

Correspondence Address: BIRCH STEWART KOLASCH & BIRCH, PO BOX 747, FALLS CHURCH, VA, 22040-0747, US

(73) Assignee: KEMIRA OYJ, Helsinki (FI)

(21) Appl. No.: 10/533,891

(22) PCT Filed: Nov. 6, 2003
(86) PCT No.: PCT/FI03/00834

§ 371 Date: Sep. 23, 2005

(30) Foreign Application Priority Data
Nov. 6, 2002 (FI) 20021986

(51) Int. Cl. A61L 2/18

(52) U.S. Cl. 422/28

(57) ABSTRACT

The invention relates to a method of inhibiting the biofilm formation by thermophilic adhering microbes of paper and board machines on the surfaces of paper or board machines, and/or removing such biofilms from the said surfaces by adding to the circulation waters of the paper or board machines at least one pure substance isolated from a plant or at least one plant extract or a mixture thereof in such a concentration which is effective against thermophilic adhering microbes. The invention further relates to a method of determining the need of the addition of an anti-biofilm agent in paper and board manufacturing processes, and an assembly kit suitable for the same.

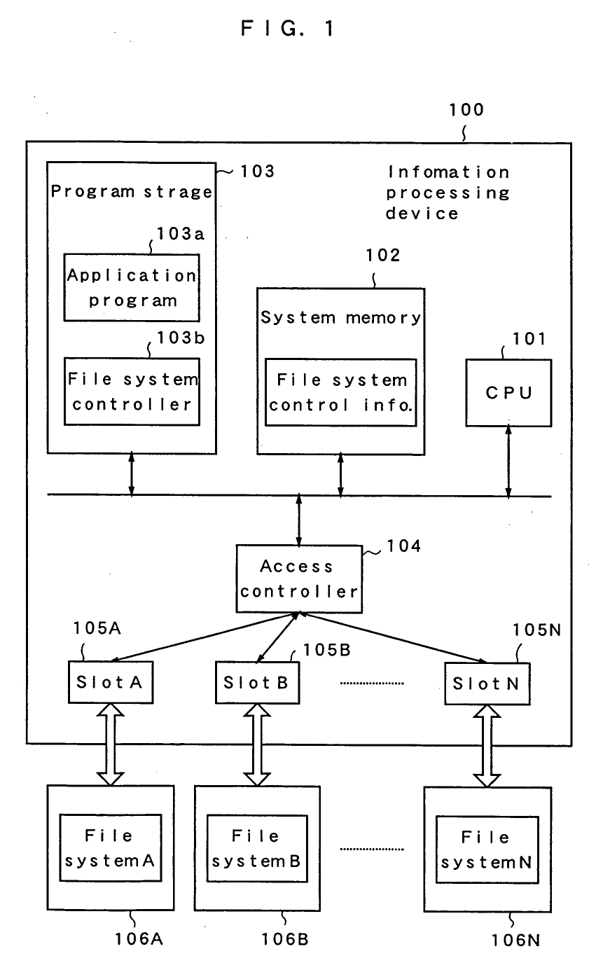

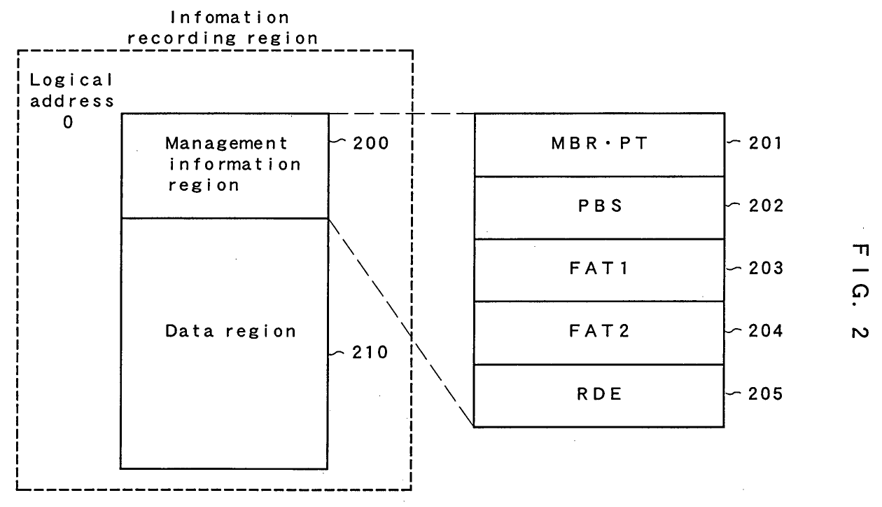

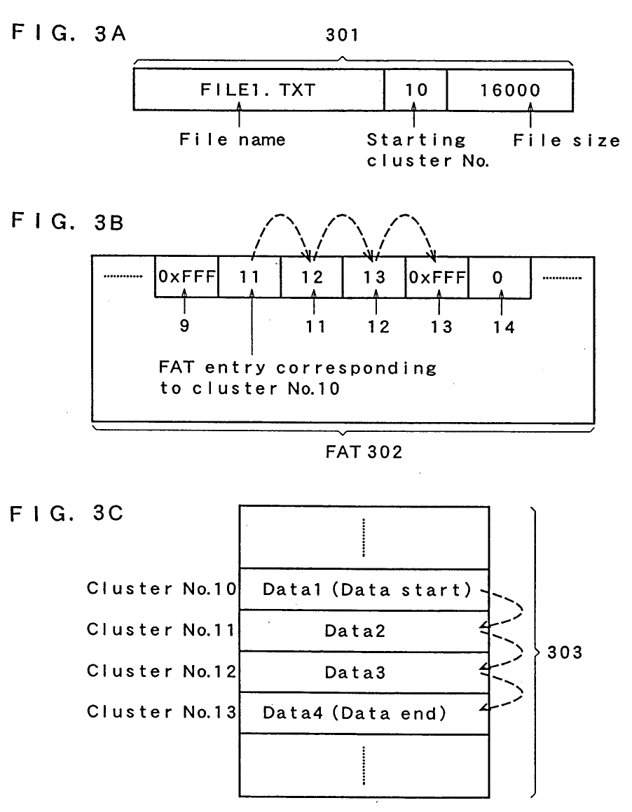

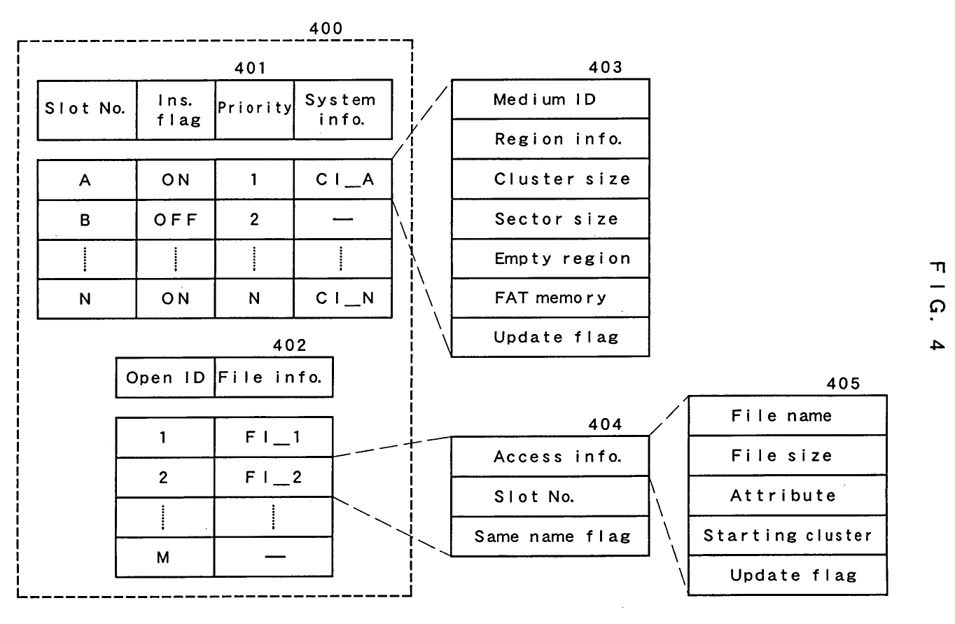

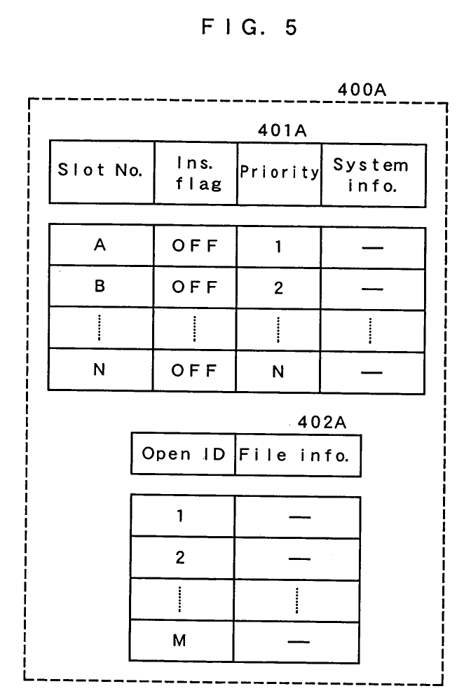

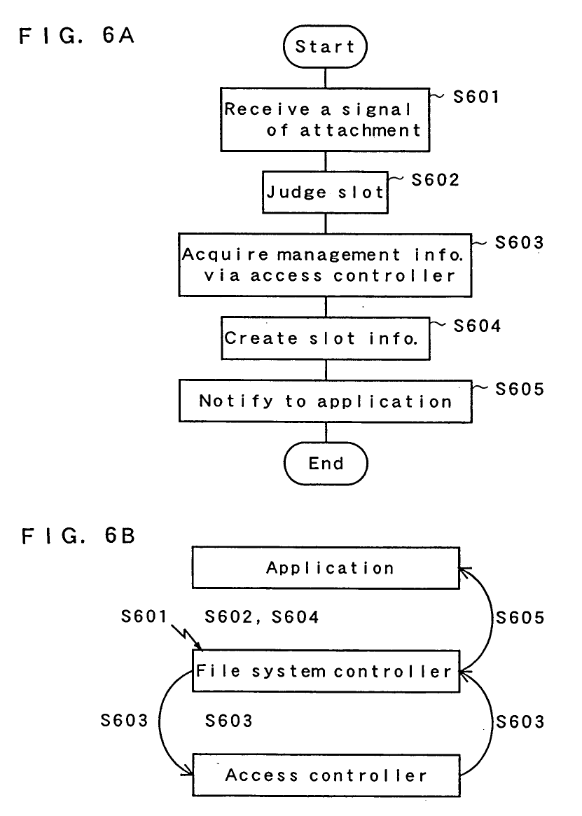

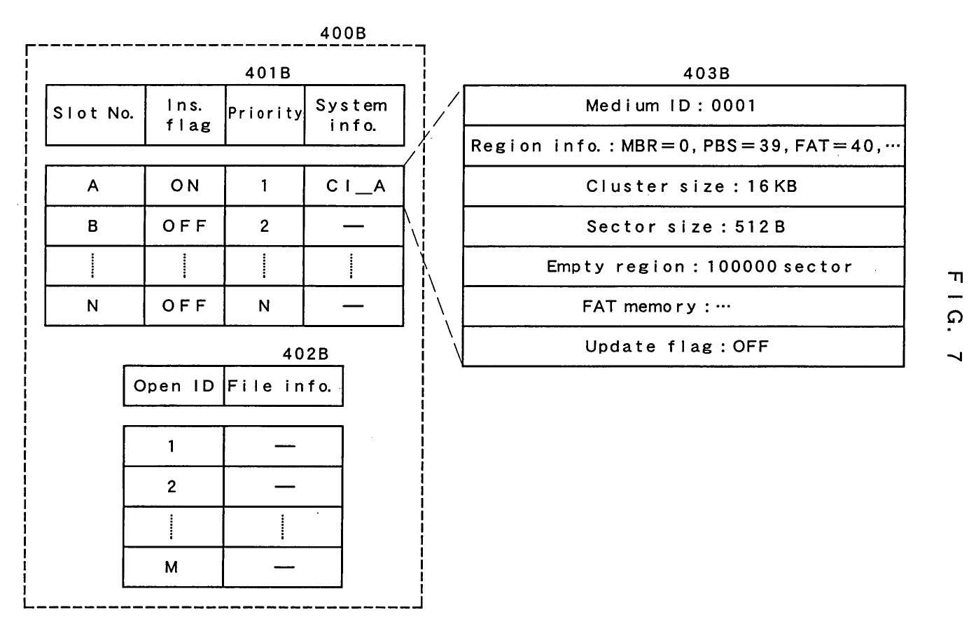

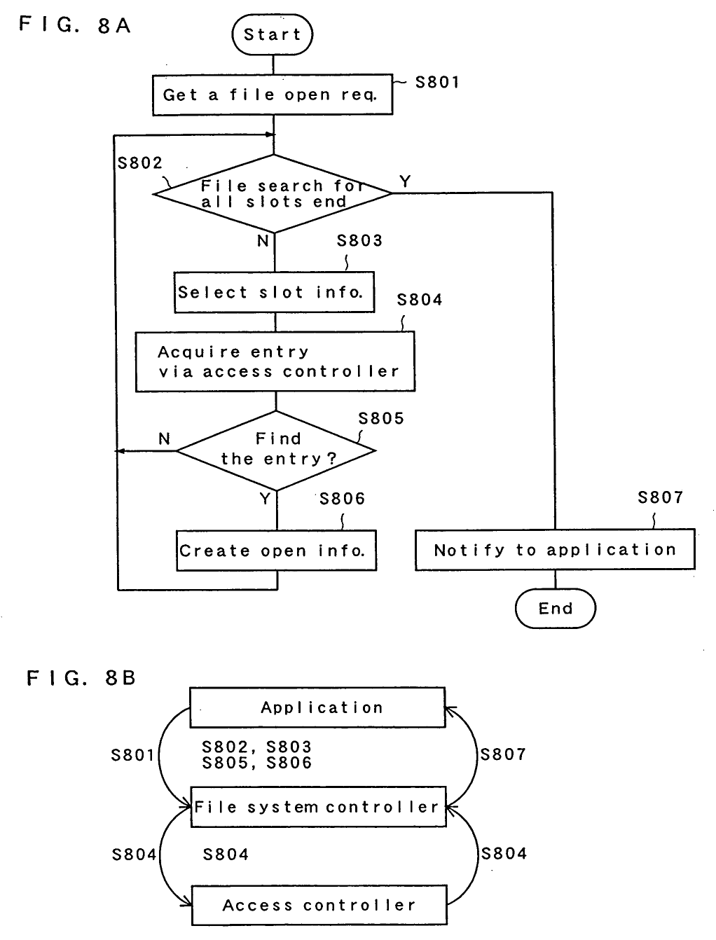

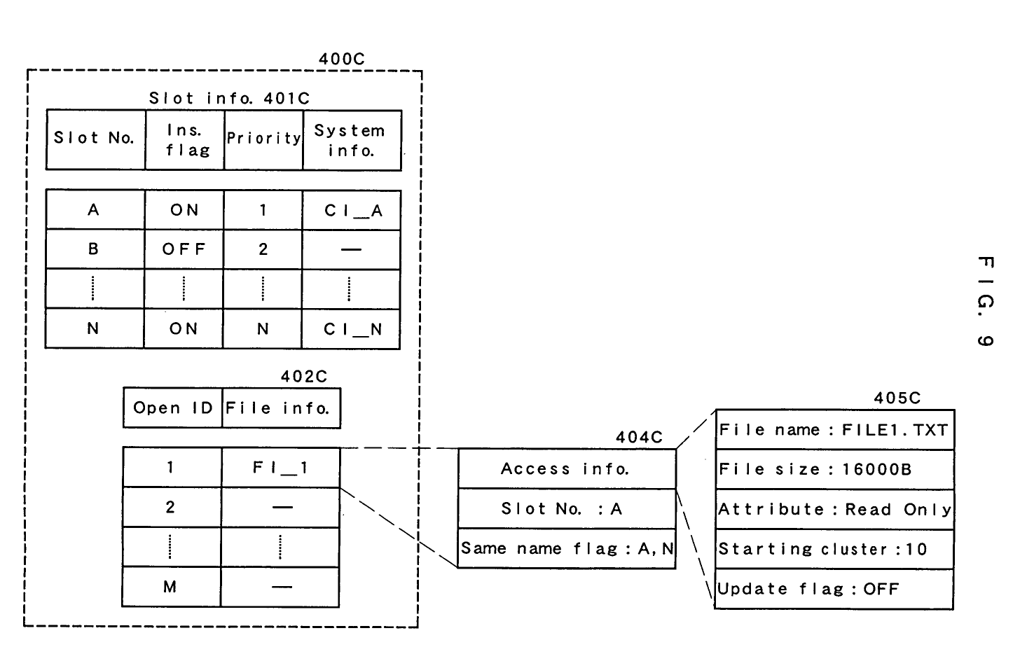

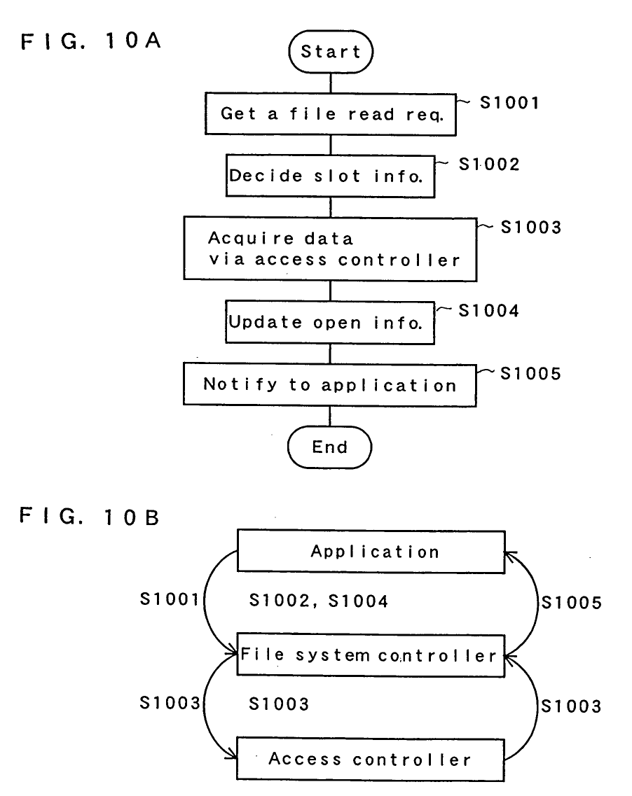

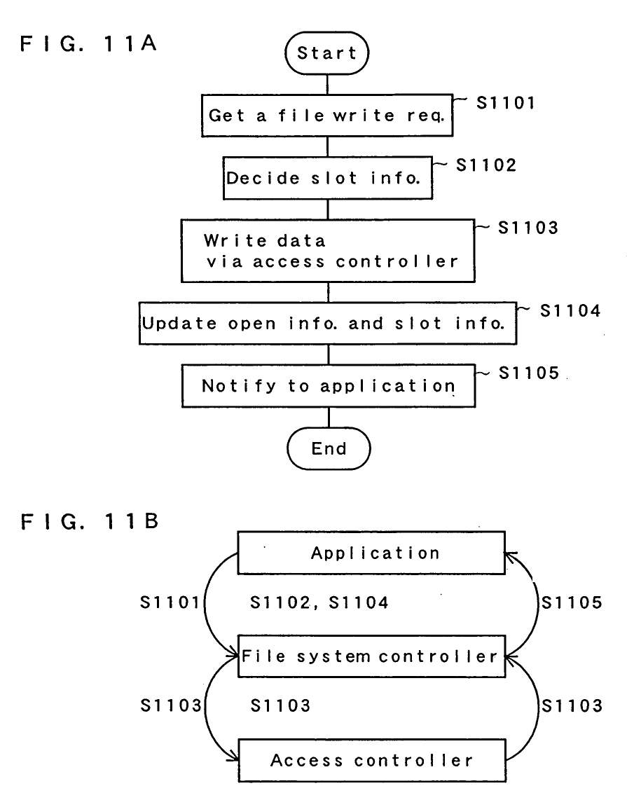

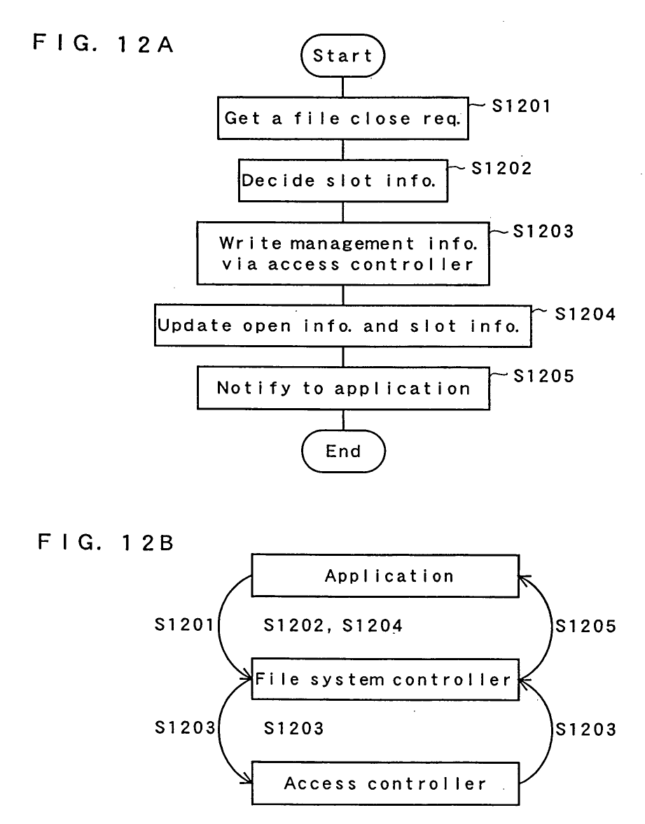

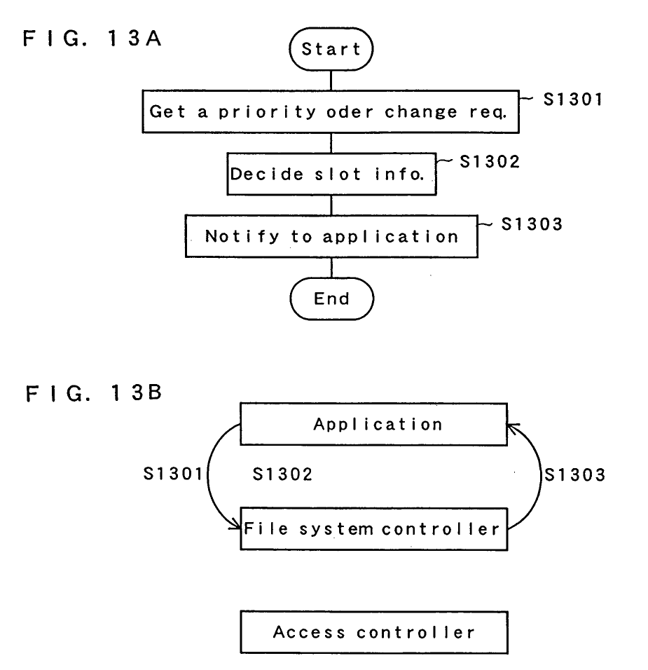

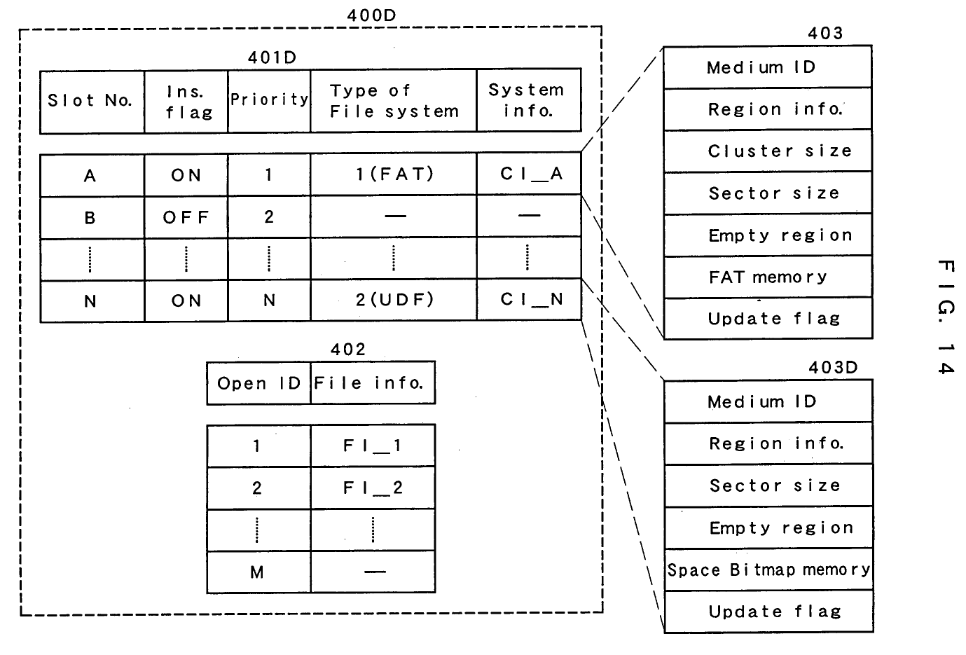

The invention relates to a method of inhibiting biofilm on the surfaces of paper and board machines, which biofilm is formed by thermophilic bacteria and/or mildew, and interferes with the process. The invention further relates to a method for determining the need for dosing an anti-biofilm agent in a paper and board making process, and an assembly kit suitable for the same.

### FIELD OF THE INVENTION AND PRIOR ART

The environment of a paper machine is favourable for the growth of various microorganisms. The paper machine water provides microbes with the nutrients they need, a suitable pH (4 to 9) and temperature (45 to 60° C.). Microbes enter the process along with raw materials, such as fibre, chemicals and water. Free-swimming microorganisms are not as harmful to the process as microbes that adhere to the surfaces of the paper machine and form biofilms. Washing the biofilms from the paper machine surfaces is difficult and often requires the use of strong chemicals. The microbes living in the biofilm are more resistant to biocides than free-swimming microbes. When spontaneously detaching from the surfaces, the biofilm deposits may block filters, cause web breaks, and impair the quality of the paper by making holes or spots, for example. Without biofouling, the runnability and, thus, the productivity of paper machines would be distinctly better than at present.

According to the newest studies (M. Kolari, J. Nuutinen and M. S. Salkinoja-Salonen, Mechanism of biofilm formation in paper machines by Bacillus species: the role of Deinococcus geothermalis, Journal of Industrial Microbiology & Biotechnology (2001), pp. 343-351), an essential factor in the biofilm formation is a so-called primary adhering bacterium (Deinococcus geothermalis), which can induce the biofilm formation. This bacterium is fairly common in paper machines. By preventing this bacterium from adhering to steel surfaces would thus reduce the biofilm formation that is harmful for the functioning of paper machines.

Marko Kolari, Academic Dissertation in Microbiology: Attachment Mechanisms and Properties of Bacterial Biofilms on Non-Living Surfaces, Dissertationes Biocentri Viikki Universitatis Helsingiensis December 2003, a doctoral thesis, the University of Helsinki, has studied, i.a. the occurrence and the mutual interactions of some biofilm-formers found in papermaking process, such as Deinococcus geothermalis, in biofilms, as well as the effect of nutrients and chemicals on these microbes. Generally, the determinations employ pure cultures of isolated microbes.

The patent publication U.S. Pat. No. 6,267,897 B discloses a method for preventing the biofilm formation in commercial and industrial water systems by adding an essential oil into the system. As examples of water systems, the publication cites, among others, cooling water, water in the food industry, systems of pulp and paper mills, pasteurizing apparatuses of breweries, fresh-water systems, etc. This patent publication describes a test, which studies the biofilm formation on glass surfaces of Sphaerotilus natans, a mesophilic mucoid micro-organism, which commonly occurs in paper mills. The test results indicate that eucalyptus oil, oil of cassia, and tea tree oil prevent the attachment of the studied bacterium on glass surfaces more effectively than the copolymer of ethylene oxide and propylene oxide that was used as a reference compound. According to this patent publication, eucalyptus oil and oil of cassia, which are commercial drug preparations, are particularly advantageous essential oils and are prepared by distillation in steam, as is well known. The other essential oils specified in this patent publication are made by distillation in steam or by compression.

The said mesophilic bacterium Sphaerotilus natans is not found in modern hot paper machines, because it cannot grow at temperatures of 50 to 60° C. Instead, a truly problematic bacterium in the paper machines of today is the Deinococcus geothermalis, which grows at high temperatures (56 to 57° C. at a maximum). Other thermophilic problematic microbes include the adhering bacteria Meiothermus silvanus, Burkholderia cepacia and Thermomonas sp. and the adhering mildew Aspergillus fumigatus.

The present invention is particularly intended to prevent such biofilm formation, which involves, as an essential part, the thermophilic problematic microbes that grow at the high temperatures (50 to 60° C.) of today's paper machines.

Accordingly, the object of the invention is to provide a method and an agent used therein, which can be used effectively for preventing the biofilm formation of thermophilic microbes on the surfaces of paper or board machines.

### DESCRIPTION OF THE INVENTION

It has now been discovered that an environmentally friendly, natural and effective biofilm prevention method can be found in compounds contained in natural plants, many of these compounds being effective against microbial growth. Such compounds are essential for the survival of plants in nature. Laboratory tests were conducted, for which 110 compounds were selected, originally isolated from plants, or synthetic derivatives of the compounds, which in other studies have been found to have biological effects, as well as 92 Finnish natural plant extracts. By using the most effective agents of these, the biofilm formation by thermophilic bacteria can be decreased, whereby it is possible to increase the output capacity of paper and board machines. According to the laboratory studies conducted, the most effective agents reduce the adherence of biofilm microbes to surfaces even by more than 90%.

Thus the invention provides, a method, which can be used to prevent the biofilm formation by thermophilic adhering microbes (bacteria and/or mildews) found in paper and board machines on the surfaces of paper and board machines, and/or which can be used to remove the already formed, harmful biofilms from the said surfaces. This method is characterized in that at least one pure substance that is isolated from a plant or at least one plant extract or a mixture thereof, is added in such a concentration to the circulation waters of paper and board machines, which is effective against thermophilic adhering microbes.

In this connection, the pure substance refers to a natural substance isolated from a plant or to a synthetic equivalent or derivative thereof and, in addition, it should be effective against biofilm-formation by thermophilic microbes on surfaces and/or be able to remove such biofilms from the surfaces. Respectively, the plant extract should be effective against biofilm-formation by thermophilic microbes on surfaces and/or be able to remove such biofilms from the surfaces. The reduction in biofilm should be at least 50%, preferably at least 70%, and most preferably at least 90%.

The said plant extract may originate from the following plants or parts of plants: Japanese rose, rosebay willow herb, meadowsweet or salvia. The plant extract can be obtained by extracting the plant or part of it with a solvent or a mixture of solvents. One preferable solvent is methanol. Other solvents suitable for the extraction include acetone, ethanol, hexane and chloroform.

The said pure substance isolated from the plant or its synthetic derivative may be a phenolic compound, such as an ester of a phenolic acid. A preferable ester of the phenolic acid is the alkyl ester of gallic acid, which preferably is octyl gallate or lauryl gallate or a mixture thereof.

The pure substance or the plant extract or the mixture thereof is added to the circulation water of the paper or board machine to a product concentration, which may be 1 to 1000 ppm, preferably 5 to 200 ppm, and most preferably 10 to 100 ppm as calculated from the dry weight of the pure substance or the plant extract.

The pure substance or the plant extract or the mixture thereof can be dosed in the circulation water of the paper or board machine either periodically, preferably 2 to 8 times a day, or as a single dose once a day. These agents or mixtures thereof can also be dosed into a container at high doses of 500 to 5000 ppm (calculated as dry matter) so as to detach the various adhering bacteria of the container surfaces by means of so-called shock dosing.

According to the invention, raw extracts prepared from the said plants, or the most effective components isolated from these raw extracts can be used.

The invention also relates to the use of the said pure substance that is isolated from the plant or of the said plant extract or of the mixture thereof for the prevention of the biofilm-formation by the thermophilic adhering microbes (bacteria and/or mildews) of the paper or board machines on the surfaces of paper or board machines and/or to the removal of such biofilms from the said surfaces.

It has now also been discovered that in papermaking processes, the presence of biofilm-forming, adhering microbes in the process can be monitored, and that the effect of the agents that prevent biofilm-formation, so-called anti-biofilm agents, on the adhering microbes in question can be determined directly from a process sample by means of a method that includes the steps of:
i) taking a sample from a papermaking process, e.g., from surfaces of the paper or board machine, a process water or raw materials,
ii) if needed, removing any loose inorganic and/or organic material from the sample, and suspending the remaining sample in an aqueous solution,
iii) shaking the suspended sample in a culturing device with a nutrient solution and, optionally, with an anti-biofilm agent for 8 to 48 h, preferably 12 to 24 h,
iv) removing the growth solution together with any material which may loose with said solution, such as any planktonic material and biofilm bacteria that are not adequately adhered, from the device and staining the microbes adhered to the wall of the device, and
v) detecting qualitatively and/or quantitatively, on the basis of the colour formation and intensity, the presence of adhering microbes in said suspended sample and, optionally, in said suspended sample treated with said anti-biofilm agent.

In this connection, the term “anti-biofilm agent” generally refers to an agent that has an activity in reducing or preventing microbe growth, especially the formation of biofilms or agglomerate caused by the adhering microbes in paper or board manufacturing processes. The term refers, i.a. to the biocidic chemicals known from papermaking and, in addition, to the plant-based agents used in the present invention, such as the pure substances that are isolated from plants, plant extracts, mixtures thereof and to the synthetic equivalents thereof.

Based on the result of the method, i.e. the colour formation and intensity, it is thus possible to determine and assess the need of the addition, i.e. dosing, of an anti-biofilm agent to the process before and/or after the anti-biofilm agent is added; in other words, whether any anti-biofilm agent should be added and/or readded to the process. Particularly preferably, the determination is effected in order to monitor the presence of biofilm-forming microbes in the process before and/or after the addition of a plant-based anti-biofilm agent according to the invention.

In a further preferable embodiment, the determination is used to select an agent, i.e., an anti-biofilm agent, preferably a pure substance isolated from a plant or a plant extract or a mixture thereof, suitable for the prevention of the biofilm formation in the process in question, and/or to define the concentration of said anti-biofilm agent needed for an effective prevention of the biofilm formation.

In the determination method (i), the sample is typically slime or biofilm/deposit taken/detached from a process water or from the walls of the equipment.

According to the invention, any loose inorganic and/or organic material is removed from the sample, for example, by means of filtering and/or washing before starting the actual test. In a preferred embodiment, the sample is, for example, a deposit sample, which is washed in step (ii) to remove any microbial material that looses easily, any planktonic microbes and any so-called secondary biofilm bacteria. Washing is preferably carried out, for example, by mixing the sample in a washing liquid, such as sterile water, by allowing the obtained solution to settle, whereby part of the sample which remains agglomerated may sediment, by removing the liquid phase above the possible sediment, and, preferably, by repeating the procedure 5 to 10 times in total. Washing can thus be used to improve the selectivity of the determination with respect to the actual problem makers, i.e., the primary biofilm formers, which are capable of adhering to and growing on clean surfaces and to which the secondary adhering bacteria can in turn be adhered to. Furthermore, the washing can be used to reduce the effect of the non-harmful planktonic microbes, for example, when determining an anti-biofilm agent that effective by prevents the biofilm formation.

In case of a sample of a process water, the sample need not to be washed.

The slime sample in the paper industry is often very viscous; therefore, the sample, preferably a sample remained after washing, is suspended in sterile water or in an aqueous solution in a known manner, e.g. in a dilution of 1:10-1:40, by mixing effectively to obtain a homogenized sample for the application thereof at stage (iii). The suspended sample is then applied into one or more recesses of a culturing device, for example, into the wells of a well-plate. In connection with the invention, it was also found that the homogenization of the samples may cause problems; therefore, especially when the determination is carried out as a series of samples, i.e. as a test serial and/or as a serial of anti-biofilm agent treatments, it is advantageous to apply the suspension into each recess in amounts not commonly used in the field, i.e. at least 1.5 ml, preferably about 2 to 10 ml, more preferably 2 to 5 ml, for example 2 to 3 ml. For that purpose e.g. commercially available well-plates of 6- or 12-wells may be used as the culturing device. If desired, test tubes or the like may also be employed.

The cultivation of the sample by shaking without and with an anti-biofilm agent is carried out in a nutrient solution suitable for the biofilm formers. Typically, at stage (ii), the sample is suspended in a nutrient solution. When needed, additional nutrient solution can then be added into the recesses. The nutrient solution can be a commercially available nutrient solution, e.g. R2 broth (commercially available, for example, Difco), or a process solution which is taken from the process and sterilized and preferably supplemented with nutrients.

In said assay, preferably, the effect of one or more of the above-mentioned plant-based anti-biofilm agents on the biofilm formers, particularly on thermophilic primary adhering microbes, is also investigated in order to find such an agent of a plant origin and/or the concentration thereof, which is/are suitable for the process.

Thus, the sample suspended at stage (iii) is subjected, e.g. in an amount of 2 to 5 ml, such as 2 to 3 ml, without any anti-biofilm agent (=0 sample) and together with each anti-biofilm agent to be tested into the recesses of the culturing device. If desired, the treatment can also be carried out with a mixture of anti-biofilm agents. The anti-biofilm agent is preferably applied in a form of a solution, at a concentration suitable for said agent, which concentration may, of course, vary considerably depending on the agent. The anti-biofilm agent/agents are preferably tested at various concentrations in accordance with the known practice, i.e. as a serial of dilutions, to determine the amount of the addition suitable for the process in question. Furthermore, in addition to the plant-based anti-biofilm agents also further agents may be tested for the use together with the plant-based agent according to the invention.

The culturing is carried out at a temperature which may vary from the ambient temperature to 65° C., preferably 35 to 65° C., more preferably 40 to 60° C., most preferably at a temperature which is close to the process temperature, from which the sample has been taken, usually in a range between 40 to 60° C. Shaking is carried out in accordance with the usual practice in the field, e.g., in a shaker, at a velocity of 100 to 300 rpm, preferably 150 to 260 rpm, at the temperatures mentioned above and for the period of time presented above.

After the culturing in a shaker (iv), the solution together with any material which looses with the solution, such as any planktonic growth and any biofilm bacteria that is not adequately adhered, is removed from the recesses. When needed, also the solution may be examined for the presence of a planktonic growth that has detached from the sample, such as from an agglomeration, and/or for the effect of the anti-biofilm agent on this growth.

After removing the solution, the recesses are typically washed, e.g., with sterile water, and the microbial component attached to the walls is stained with a staining agent in accordance with the known practice. Staining can thus either be carried out using (i) stains, e.g., crystal violet or safranine, that indicate the total amount of biomass (ii) stains such as acridine orange, etidium bromide, DAPI, SYTO16 or other nucleic acid colours, that indicate the number of cells in the microbes (iii) stains for example, LIVE/DEAD™, CTC or different tetrazolium compounds, that indicate the liveliness of the microbe cells, or (iv) specific enzyme substrates that indicate the enzymic activity and turn into fluorescent compounds in case the biofilm comprises, e.g., starch degrading activity, chitinase activity, esterase activity, degrading activity for lipid esters, or phosphatase activity. Any superfluous staining agent is rinsed and the colour change and intensity caused by the stained microbes are detected qualitatively, e.g. visually, and/or quantitatively, such as by dissolving the staining agent e.g. in ethanol and by detecting the intensity of the colour by means of spectrophotometry using devices well known in the art, such as the absorbance reader of well-plates, or by a fluorometer.

On the basis of the results obtained, it is possible to determine the need of the application of an anti-biofilm agent and the type of an anti-biofilm agent which is effective, as well as the concentration which was effective for the sample, and thus effective against the adhering microbes present in the process.

The determination method according to the invention enables a quick detection of the presence of adhering bacteria in a process of papermaking industry, and of the anti-biofilm agent effective against said bacteria (the need and the amount of the addition), whereby the delay between the sampling and detecting a problem and the start-point of measures to overcome the problem becomes shorter compared with the traditional determinations which take several days and are based on pure cultures and, furthermore, which often merely determine the prevention of the growth of planktonic microbes.

The invention further provides an assembly kit for determining the need of the addition of an anti-biofilm agent. The kit comprises
a) a pre-treatment device, e.g., a plastic test tube provided with a cap, for taking a sample and for removing any loose organic and/or organic material from the sample,
b) optionally, a mixer, such as a vortex mixer, for suspending/homogenizing the sample,
c) a culturing device provided with a plurality of separate recesses, the volume of each recess being at least 2 ml, preferably at least 3 ml, and also larger than the volume of the sample dose subjected into the recess and comprising the suspended sample in an amount of at least 1.5 ml, preferably 2 to 10 ml, more preferably 2 to 5 ml, and, optionally, an anti-biofilm agent, such as a solution of an anti-biofilm agent, and/or an additional nutrient solution,
d) a shaker, preferably a thermal shaker, to shake the culturing device for enabling the formation of biofilm,
e) reagents, which include
   a. at least one anti-biofilm agent, such as a solution of an anti-biofilm agent, optionally as a serial of dilutions, for treating the suspended sample during shaking,
   b. a sterile nutrient solution, and
   c. a solution of a staining agent for staining the microbes adhered to the recess.

The culturing device is naturally selected in accordance with the volume of the sample dosage used in the method, so that its recesses accommodate the desired dosages of the suspended sample and, optionally, the anti-biofilm agent solution and/or the nutrient solution that is further added, and that the dosed solution remains in the recess for the time of shaking. As an example, the commercially available well-plate of 6- or 12-wells may be mentioned.

The assembly kit can further include a detecting device, such as the one mentioned above, for the qualitative and/or quantitative detection of the stained adhering microbes, sterile water for washing the sample, metering devices, such as pipettes, for applying the suspended sample and the washing, nutrient and/or anti-biofilm agent solutions.

Furthermore, the reagents of the kit may be in multidose or single-dose packages, or some reagents, such as the anti-biofilm agent and/or the additional nutrient solution can be prefilled in the wells of the culturing device, such as the well-plate, whereby the wells are sealed with a removable film, for example.

In the following, the invention is described in detail with reference to laboratory research and examples.

Studies Conducted

1. Ability of Pure Substances to Prevent Biofilm Formation by Adhering Bacteria

The effect of pure substances on the biofilm formation by the adhering bacteria isolated from paper machines, such as Deinococcus geothermalis E50051, Burkholderia cepacia F28L1, Thermomonas sp. 11306 and Meiothermus silvanus R2A-50-3 was studied by means of a 96-well plate test (a well-plate of polystyrene, cell culture grade, hydrophilic). The bacteria had been inoculated from dishes into nutrient liquor tubes 24 h earlier and grown in agitation at 45° C. At the beginning, 2.5 μl of a pure substance dilution (dissolved in dimethyl sulphoxide, DMSO) were pipeted into the wells in two different concentrations. After this, a bacterium suspension was added, which had been diluted to about 2% with an R2 nutrient broth (pH 7) 250 μl/well. The R2 broth is a synthetic culture medium that is well suited for the cultivation of paper machine adherers. The final concentrations of the pure substances in the wells of the well-plates were 25 μmol 1⁻¹ or 250 μmol 1⁻¹. The plates were incubated in agitation at 45° C. at 160 rpm for 17 to 18 h. After cultivation, the well-plates were emptied, rinsed carefully with tap water, and a 0.1% SDS solution (an anionic surfactant) was added into the wells in an amount of 280 μl/well. The plates were again placed into the shaker for 1 hour. Thus, the results show both the agents that prevented the biofilm formation and those that lead on to forming a biofilm having structure so loose that it came off in washing, which normally does not affect the biofilms of the adhering bacteria in question. After washing with SDS, the plates were rinsed with tap water. The biofilms were stained with a crystal violet solution and rinsed again. For reading the results, the colour attached to the biofilm was dissolved in 96% ethanol and the absorbance of the solutions in the wells was measured by means of an ELISA reader at a wavelength of 595 nm. By comparing the results with the wells treated with DMSO only, the percentage of biofilm inhibition could be calculated for each pure substance.

### EXAMPLE 1

The number of pure substances that were studied totalled 110. Table 1 shows the nine pure substances that had a strong anti-biofilm effect against more than one adhering bacterial strain (a reduction of biofilm of more than 50% for more than one of the strains tested).

Three pure substances (lauryl gallate, octyl gallate and nordihydro quaiaretic acid) had a broad-spectrum anti-biofilm effect, as they decreased the biofilm formation of all four different adhering bacteria in the concentration of 250 μmol 1⁻¹. The gallates, especially the lauryl gallate, were also effective in the content of 25 μmol 1⁻¹. The molecular weight of the lauryl gallate is 338.45 g mol⁻¹, i.e., the substance had a broad-spectrum anti-biofilm effectiveness at a content of 8.5 mg 1⁻¹ (=8.5 ppm). The octyl and lauryl gallates decreased the adherence of biofilm bacteria to the surfaces by more than 90% at best in a lean nutrient solution (in the content of 250 μM against Deinococcus geothermalis and Burkholderia cepacia).

Many pure substances were found to have a distinct inhibiting effect in the content of 250 μM, but it was observed that a lower content of 25 μM, in turn, increased the biofilm formation (e.g., coumarin 102, flavone and nordihydro quaiaretic acid in Table 1). This could be interpreted so that, at the content of 25 μM, these substances do not yet have a sufficiently high active ingredient content to prevent biofilm formation, but it is enough to make the free-swimming form of growth unfavourable, and thus may help the bacteria to gravitate towards the biofilm.

TABLE 1
Pure substances which inhibited biofilm formation by adhering bacteria.

| | | Reduction percentage of biofilm formation¹ | | | |
|---|---|---|---|---|---|
| | | Adhering bacterium | | | |
| | Content | Deinococcus | Burkholderia | Meiothermus | Thermomonas |
| Pure substance | μM | geothermalis | cepacia | silvanus | sp. |
| Coumarin 102 | 250 | 97 | −19 | 87 | 86 |
| | 25 | 49 | −20 | −38 | −57 |
| Coumarin 106 | 250 | 99 | −85 | 95 | 91 |
| | 25 | −16 | −11 | 82 | 14 |
| (-)-Epigallo- | 250 | −11 | 69 | 66 | −16 |
| cathechin gallate | 25 | −93 | 18 | 79 | −27 |
| Flavone | 250 | 24 | −33 | 90 | 84 |
| | 25 | 24 | −6 | −164 | −56 |
| Lauryl gallate | 250 | 95 | 96 | 78 | 77 |
| | 25 | 97 | 64 | 82 | 81 |
| 2′-methoxy-alpha- | 250 | 75 | −41 | 89 | 32 |
| naphto-flavone | 25 | 11 | −38 | 53 | 3 |
| Nordihydro | 250 | 85 | 20 | 89 | 44 |
| guaiaretic acid | 25 | −23 | −68 | 92 | 7 |
| Octyl gallate | 250 | 94 | 99 | 85 | 71 |
| | 25 | 96 | −20 | 63 | 73 |
| Silybine | 250 | 89 | −121 | 93 | −98 |
| (silymarine) | 25 | 18 | −44 | 80 | −9 |

¹Calculated from the A₅₉₅ values and compared with wells that were treated with DMSO only.

### EXAMPLE 2

Further studies were conducted on the best anti-biofilm agents of Example 1 by including in the test several bacterial strains, and also testing without SDS washing.

The adhering bacterial strains E-1vk-R2A-1 and E-jv-CTYE3, which were isolated from the paper machine and not yet identified, and the Aspergillus fumigatus mould G3.1 were included. The reference substance used was the commonly used biocide Fennosan M9, whose effective ingredient is methylene bisthiocyanate (9%). The results are shown in Table 2.

The gallates proved to also be effective against the new adherers. They were also effective without SDS washing, which leads to the conclusion that the influencing mechanism of the substances comprises the inhibition of biofilm formation. Both the lauryl and the octyl gallates were active against most test microbes even at the content of 25 μM. They were also effective against the B. cepacia and Thermomonas biofilms that are difficult to control. Surprisingly, the effect of lauryl gallate in the R2 broth tests was better with a low content than with a high content. This may be a consequence of the poor solubility of the substance in the R2 broth. The effect of lauryl gallate in the content of 25 μM (8.5 ppm) was almost on the level of the methylene bisthiocyanate (10 ppm) that was used as a reference substance.

TABLE 2
Anti-biofilm effect of the best pure substances (in DMSO) in R2 broth without SDS washing
Reduction percentage¹ of biofilm formation
Adhering bacterium and mould

| | | | | | | |
|---|---|---|---|---|---|---|
| D. geothermalis | B. cepacia | Thermomonas sp. | M. silvanus | E-lvk-R2A-1 | E-jv-CTYE3 | G 3.1. |

pure substance content (μM)

| | | | | | | | | | | | | | | |
|---|---|---|---|---|---|---|---|---|---|---|---|---|---|---|
| Pure substance | 250 | 25 | 250 | 25 | 250 | 25 | 250 | 25 | 250 | 25 | 250 | 25 | 250 | 25 |
| Lauryl gallate | 24 | 91 | 46 | 84 | 53 | 95 | 33 | 94 | −125 | 92 | 87 | 98 | 25 | 55 |
| Octyl gallate | 77 | 69 | 93 | −49 | 79 | 90 | 87 | 96 | 63 | 90 | 96 | 98 | 97 | 57 |
| Nordihydro guaiaretic acid | 77 | −8 | 91 | −45 | 86 | −6 | 93 | 95 | 86 | 57 | 98 | 78 | 55 | −11 |
| Coumarin 102 | 93 | −15 | −147 | −33 | 98 | −12 | 98 | 18 | 91 | −96 | 97 | 34 | 80 | −11 |
| 2′-methoxy-□-naphto-flavone | 4 | 2 | −31 | 46 | 42 | 56 | 98 | 56 | 90 | −101 | 99 | 76 | 61 | 59 |

Content of biocide (ppm²)

| | | | | | | | | | | | | | | | | | | | | | |
|---|---|---|---|---|---|---|---|---|---|---|---|---|---|---|---|---|---|---|---|---|---|
| | 30 | 20 | 10 | 30 | 20 | 10 | 30 | 20 | 10 | 30 | 20 | 10 | 30 | 20 | 10 | 30 | 20 | 10 | 30 | 20 | 10 |
| M9 | 99 | 99 | 98 | 93 | 93 | 91 | 98 | 99 | 99 | 100 | 99 | 99 | 100 | 100 | 100 | 98 | 98 | 98 | 100 | 100 | 100 |

¹As calculated from A₅₉₅ values and compared with wells treated with DMSO only
²Content of the active ingredient methylene bisthiocyanate

### EXAMPLE 3

The anti-biofilm effect of the most effective pure substances of Example 1 was also studied in paper machine water cultivation (white water, 1 g/l of starch and 300 mg/l of a yeast extract was added, sterilized, 250 μl/cup, pH 7, inoculation 2%, growing 48 h, 45° C., 160 rpm). The results are shown in Table 3. Octyl and lauryl gallates decreased the adhesion of biofilm microbes to the surfaces by more than 90% at best in sterilized white water (in a content of 25 μM against Meiothermus silvanus and the adhering bacterium E-jv-CTYE3). In order for the adhering bacteria to form biofilm in paper machine water, a longer cultivation time is required. The effect of both the pure substances and that of M9 remained minor, perhaps because of the longer time of cultivation. In the paper machine environment, this problem does not exist, as the active ingredient is added into the process at regular intervals.

TABLE 3
Anti-biofilm effect of the best pure substances (in DMSO) in white water without SDS washing
Reduction percentage¹ of biofilm formation¹
Adhering bacterium and mould

| | | | | | | |
|---|---|---|---|---|---|---|
| D. geothermalis | B. cepacia | Thermomonas sp. | M. silvanus | E-lvk-R2A-1 | E-jv-CTYE3 | G 3.1. |

pure substance content (μM)

| | | | | | | | | | | | | | | |
|---|---|---|---|---|---|---|---|---|---|---|---|---|---|---|
| Pure substance | 250 | 25 | 250 | 25 | 250 | 25 | 250 | 25 | 250 | 25 | 250 | 25 | 250 | 25 |
| Lauryl gallate | 83 | 90 | 48 | −5 | 71 | −1 | 81 | 95 | 28 | 54 | 81 | 91 | −18 | 26 |
| Octyl gallate | 84 | 1 | 46 | −12 | 53 | 8 | 84 | 98 | 37 | 85 | 86 | 87 | 9 | 42 |
| Nordihydro guiaretic acid | 91 | −36 | −19 | −25 | 32 | −23 | 90 | 72 | 58 | 82 | 92 | 1 | −19 | 12 |
| Coumarin 102 | 99 | −9 | −8 | −7 | 79 | 22 | 98 | 40 | 91 | −28 | 98 | 13 | 68 | 29 |
| 2′-methoxy-□-naphto-flavone | −2 | −2 | 76 | 5 | 28 | 18 | 90 | 70 | 90 | −16 | 95 | 66 | 62 | 54 |

content of biocide (ppm²)

| | | | | | | | | | | | | | | | | | | | | | |
|---|---|---|---|---|---|---|---|---|---|---|---|---|---|---|---|---|---|---|---|---|---|
| | 30 | 20 | 10 | 30 | 20 | 10 | 30 | 20 | 10 | 30 | 20 | 10 | 30 | 20 | 10 | 30 | 20 | 10 | 30 | 20 | 10 |
| M9 | 100 | 100 | 99 | 57 | 53 | 58 | 68 | 72 | 66 | 96 | 98 | 95 | 93 | 99 | 96 | 98 | 100 | 99 | 100 | 100 | 100 |

¹As calculated from A₅₉₅ values and compared with wells treated with DMSO only
²Content of the active ingredient methylene bisthiocyanate

2. Ability of Plant Extracts to Prevent Biofilm Formation by Adhering Bacteria

The 96-well plate test (a cup plate of polystyrene, cell culture grade, hydrophilic, R2 liquor 250 μl/well, pH 7, agitation at 160 rpm, 45° C., 17 to 18 h) described above was use as the testing method. The adhering bacteria of the paper machines studied comprised Deinococcus geothermalis E50051, Burkholderia cepacia F28L1, Thermomonas sp. 11306 and Meoothermus silvanus R2A-50-3. The final contents of the plant extracts (extracted with methanol) in the wells were 20 or 200 mg 1⁻¹. After the cultivation, the plates were rinsed and washed with 0.1% SDS (an anionic surfactant) at agitation of 120 rpm for 1 h. The wells of the plates were rinsed with tap water and stained with crystal violet. For reading the results, the colour adhered to the biofilm was dissolved in 96% ethanol and the absorbance of the well solutions was measured with the ELISA reader at a wave length of 595 nm.

### EXAMPLE 4

The test method described above was employed to study the preventive effect of 92 plant extracts on biofilm formation by adhering bacteria. Table 4 shows only the plant extracts made by methanol (18 samples) that prevented more than one bacterium. The best plant extracts were the flower of the sheep's sorrel, the flower of the yellow loosestrife, the leaf of the small-flowered hairy willow herb, the flower of the small-flowered hairy willow herb, the root of the large-flowered hemp-nettle, the lead of the Japanese rose, the stem of the Japanese rose, the petal of the Japanese rose and the leaf of sage.

Some of the most interesting plants included Japanese rose, the small-flowered hairy willow herb and salvia, all of which had the broadest-spectrum anti-biofilm effect. Furthermore, all aerial parts of Japanese rose and the small-flowered hairy willow herb that were studied showed an anti-biofilm effect. The extracts made of Japanese rose were the only ones that also had an effect on the B. cepacia biofilms that proved to be the most difficult ones to control. Regarding the plants that were found to be effective, the Japanese rose and sage would also be the easiest to grow.

TABLE 4
Methanol extracts (now in DMSO) that showed anti-biofilm effect
by adhering bacteria in cultivation in R2 broth.
Reduction percentage of biofilm formation¹
Adhering bacterium

| Deinococcus geothermalis | Burkholderia cepacia | Meiothermus silvanus | Thermomonas sp. |
|---|---|---|---|
| E50051 | F28 L1 | R2A-50-3 | 11306 |

| | Content of plant extract (mg¹⁻¹) | | | | | | | |
|---|---|---|---|---|---|---|---|---|
| Plant extract | 200 | 20 | 200 | 20 | 200 | 20 | 200 | 20 |
| Stem of garlic, organic | −39 | 3 | −127 | −36 | 40 | 30 | 58 | 51 |
| Stem of garlic | −29 | 9 | −105 | −52 | 27 | 26 | 56 | 52 |
| Sheep's sorrel, flower | 76 | −4 | 2 | −5 | 86 | 2 | 61 | −133 |
| Onion, organic | −3 | 18 | −34 | 7 | 9 | 26 | 50 | 52 |
| Common toadflax, leaf | −10 | 2 | −5 | −3 | 56 | −35 | 23 | 27 |
| Yellow loosetrife, flower | 98 | 17 | 30 | −10 | 90 | 64 | 80 | |
| Bracken, leaf | −35 | 19 | −17 | −14 | 95 | 25 | −95 | −9 |
| Mugwort, leaf | 89 | −5 | −16 | −14 | 97 | 85 | −32 | −7 |
| Small−flowered hairy willow herb, leaf | 89 | 74 | −17 | 42 | 96 | 95 | 44 | −44 |
| Borecole, organic | −31 | 0 | −91 | −7 | 23 | 22 | 51 | 56 |
| Small−flowered hairy willow herb, flower | 98 | 51 | −12 | 51 | 97 | 95 | −12 | −21 |
| Large−flowered hemp-nettle, root | 96 | 52 | 1 | 59 | 95 | 92 | 45 | −71 |
| Cabbage, organic | −51 | −1 | −114 | −33 | 19 | 28 | 52 | 42 |
| Japanese rose, leaf | 79 | 29 | 75 | 61 | 94 | 91 | 73 | −87 |
| Japanese rose, stem | 96 | 31 | 43 | −5 | 31 | | −142 | −95 |
| Autumn carrot | −15 | −12 | 12 | 14 | 19 | 18 | 56 | 49 |
| Salvia, leaf | 96 | −21 | −62 | −16 | 99 | 66 | 84 | 13 |
| Japanese rose, petal | 99 | 22 | 58 | 36 | 67 | −56 | 17 | −84 |

¹Calculated from A₅₉₅ values, compared with wells treated with DMSO only (%)

### EXAMPLE 5

The study was continued by conducting repeat extractions and tests on Japanese rose, small-flowered hairy willow herb and salvia (preserved in dry form for 2 years) for ensuring the activity. In addition, a decision was made to extract and test their kindred plants the rosebay willow herb (leaves, flowers and roots) and the meadowsweet (leaves, flowers and roots). Cognate plats often contain similar compounds and, therefore, it was assessed that also their extracts would be active. The small-flowered hairy willow herb is a relatively rare plant; therefore, we hoped that the considerably more common rosebay willow herb would also be active. The tests were conducted both with and without SDS washing; therefore, the results show the influencing mechanism of the extracts (H=weakens the biofilm, no inhibition of biofilm was perceivable without SDS washing, E=prevents biofilm formation, SDS washing showed no effect.)

The results are shown in Table 5. The best ones of the plant extracts studied (Japanese rose, rosebay willow herb, meadowsweet and sage) prevented various adhering bacteria from attaching to the surfaces, particularly D. geothermalis and M. silvanus. On the basis of the results, the new Japanese rose extracts were also active, although not quite as active as the original extracts. This may be explained by the fact that almost 2 years had already past since the plants were collected, and the contents of active ingredients may have decreased during storage. This was also the case for the small-flowered hairy willow herb. Rosebay willow herb and meadowsweet, the kindred plants of the former, were brought out as new and interesting plants. The new salvia extract was about as effective as the old one. In the repetition studies, Thermomonas sp. proved to be the most difficult of the adhering bacteria to prevent: of the extracts studied only salvia prevented it from growing.

TABLE 5
Anti-biofilm effect of new extracts of Japanese rose, small-flowered hairy willow herb and salvia,
as well as their kindred plants in R2 broth (methanol extracts, now in DMSO).
The plants had been stored in dry form for 2 years before extraction
Reduction percentage of biofilm formation¹
Adhering bacterium

| | | | |
|---|---|---|---|
| Deinococcus geothermalis | Burkholderia cepacia | Thermomonas sp. | Meiothermus silvanus |

| | Content of plant extract (mg 1⁻¹) | | | | | | | |
|---|---|---|---|---|---|---|---|---|
| Plant extract | 200 | 20 | 200 | 20 | 200 | 20 | 200 | 20 |
| Meadowsweet, root | 0 | −1 | 66 (E) | 39 | −88 | −127 | 94 (E) | 67 |
| Rosebay willow herb, leaf | 91 (E) | −13 | 60 (H) | 50 | −123 | −104 | 96 (E) | 79 |
| Rosebay willow herb, flower | 92 (E) | −18 | 55 (E) | 33 | −89 | −62 | 95 (E) | 74 |
| Meadowsweet, flower | 90 (E) | −30 | 72 (E) | 36 | −75 | −57 | 94 (E) | 74 |
| Japanese rose, other parts of flower | 59 (E) | −23 | 64 (H) | 22 | −101 | −115 | 94 (E) | 72 |
| Small-flowered hairy willow herb, leaf | 92 (E) | −21 | 15 (H) | 21 | −91 | −59 | 95 (E) | 73 |
| Meadowsweet, leaf | 92 (E) | −28 | −3 | −1 | −135 | −24 | 95 (E) | 74 |
| Rosebay willow herb, root | 66 (E) | −28 | 49 (H) | 10 | −102 | −54 | 95 (E) | 70 |
| Japanese rose, leaf | 93 (E) | −17 | 11 (H) | −8 | −133 | 1 | 93 (E) | 64 |
| Japanese rose, petal | 92 (E) | −19 | 64 (H) | 17 | −86 | −16 | 93 (E) | 68 |
| Japanese rose, stem | 85 (E) | −15 | 76 (H) | 39 | −100 | −114 | 94 (E) | 62 |
| Salvia, leaf | 81 (E) | −37 | −76 | −33 | 66 (E) | −10 | 99 (E) | 83 |

¹Calculated from A₅₉₅ values, compared with wells treated with DMSO only (%)
²Explanations of the letters behind the reference number: H = weakens the biofilm, no biofilm inhibition perceivable without SDS washing, E = prevents biofilm formation, SDS washing shows no effect

### EXAMPLE 6

The most effective ones of the extracts tested in the R2 broth were also tested in paper machine water (white water, 1 g/l of starch and 300 mg/l added, sterilized). More microbe strains were included in the test (the bacterial strains E-1vk-R2A-1 and E-jv-CTYE3 that were isolated from the paper machine and not yet identified, and the Aspergillus fumigatus mould G 3.1.) The testing method was the same well plate test (well-plate of polystyrene, cell culture grade, hydrophilic, paper machine water 250 μl/cup, pH 7, 45° C., 160 rpm for 48 h). As the bacteria do not form biofilm in the paper machine water as quickly as in the R2 broth, a longer cultivation time is necessary. The results are shown in Table 6, which indicates that the effect of the extracts remained lower than that in the R2 broth, perhaps namely because of the longer cultivation time. In the paper machine environment, this can be corrected by adding the active ingredient at regular intervals.

According to the tests conducted, the most viable plant extracts for the inhibition of harmful biofilms in paper and board machines are, thus, the Japanese rose, meadowsweet, rosebay willow herb and salvia extracts. The small-flowered hairy willow herb extracts were also effective but difficult to obtain because of their rarity.

TABLE 6
Anti-biofilm effect of the most effective plant extracts in white water (methanol extracts, now in DMSO)
Reduction percentage of biofilm formation¹
Adhering bacterium and mould

| | | | | |
|---|---|---|---|---|
| D. geothermalis | Thermomonas sp. | M. silvanus E-lvk-R2A-1 | E-jv-CTYE3 | G 3.1. |

| | Content of plant extract (mg 1⁻¹) | | | | | | | | | | | |
|---|---|---|---|---|---|---|---|---|---|---|---|---|
| Plant extract | 200 | 20 | 200 | 20 | 200 | 20 | 200 | 20 | 200 | 20 | 200 | 20 |
| Rosebay willow herb, leaf | 1 | −2 | −42 | −41 | 76 | 57 | 63 | −11 | 89 | 60 | 20 | 29 |
| Rosebay willow herb, flower | −3 | −6 | −33 | 22 | 36 | 57 | 61 | −2 | 89 | 53 | 39 | 49 |
| Meadowsweet, flower | −4 | −3 | −7 | 27 | 50 | 49 | 76 | 2 | 92 | 51 | 30 | 37 |
| Small-flowered hairy willow herb, leaf | −1 | −4 | −37 | 0 | 65 | 68 | 37 | −6 | 84 | 74 | −53 | −50 |
| Meadowsweet, leaf | 97 | −1 | −10 | 18 | 49 | 53 | 89 | −33 | 98 | 49 | 43 | 30 |
| Japanese rose, leaf | 98 | 2 | −12 | 25 | 63 | 56 | 89 | −42 | 99 | 27 | −30 | 47 |
| Japanese rose, petal | −2 | −2 | −8 | −10 | 65 | 34 | 88 | −8 | 97 | 37 | −16 | 47 |
| Japanese rose, stem | −3 | −2 | −12 | 19 | 44 | 31 | 78 | −18 | 94 | 34 | 18 | 55 |
| Salvia, leaf | 98 | 3 | 95 | 15 | 96 | 69 | 98 | 73 | 99 | −16 | −220 | −121 |

¹Calculated from A₅₉₅ values, compared with wells treated with DMSO only (%)

3. Monitoring the Presence of Biofilm-Forming Adhering Bacteria and Determing the Effect of Anti-Biofilm Agents

### EXAMPLE 7

A sample of deposit is taken from the surface of the paper machine (a disk filter) into a sample can. Sterile water is added to the slime sample and mixed intensively by means of a vortex mixer. The sample is allowed to settle and the supernatant is removed. The procedure is repeated to obtain a total of 10 washing times. Finally, the sample is diluted in an R2 nutrient solution (Difco) so as to obtain a 1:10-1:40 dilution, and is homogenized. 2 ml of the suspension thus obtained are applied into well-plates of 12 wells (N-150628 F12 12-well plates, Nunc). Part of the wells contain the sample suspension only and into the other part of the wells also the anti-biofilm agents are added into some: 1) a commercial product containing glutaraldehyde and 2) a commercial product containing DBNPA, both in two concentrations, 100 ppm and 300 ppm, each substance/concentration into a separate well, to study the effect of the anti-biofilm agent treatment. The cup plates are placed in a commercial thermal shaker and agitated for 24 h at 44° C. at a shaking speed of 160 rpm or 250 rpm. The amount of the free-floating planktonic growth is assessed by means of examining the cloudiness of the solutions.

The solution is clear in cups, into which the Fennosan GL10 anti-biofilm agent was added, indicating that the substance also affects planktonic growth. After this, the solution is removed from the cups; the cups are rinsed with tap water and filled with a safran colour, which is allowed to work for 5 min. The dye solution is removed and the cups are rinsed several times (4×), and the cups are then filled with ethanol and the dye is allowed to dissolve in ethanol for 1 h, after which the amount of the fluorescent ink is measured by a Fluoroscan device. On the basis of the test, there are adhering bacteria on the location of the sample and, in addition, both anti-biofilm agents used also decrease the adhesion of the adhering bacteria to the surfaces of the cups.

1. A method for inhibiting the biofilm formation by thermophilic adhering microbes of paper and board machines on the surfaces of paper and board machines and/or for removing such biofilms from said surfaces, characterized in that at least one pure substance that is isolated from a plant or at least one plant extract or a mixture thereof is added in such a concentration to the circulation waters of paper and board machines, which is effective against thermophilic adhering microbes.

2. The method according to claim 1, characterized in that the plant extract originates from any of the following plants or parts of them: Japanese rose, rosebay willow herb, salvia or meadowsweet.

3. The method according to claim 1 or 2, characterized in that the plant extract is obtained by extracting the plant or a part thereof with methanol, ethanol, acetone, hexane, chloroform or a mixture thereof.

4. The method according to claim 1, characterized in that the pure substance is a natural ingredient isolated from a plant, or a synthetic equivalent or derivative thereof.

5. The method according to claim 4, characterized in that the pure substance is a phenolic compound, preferably an ester of a phenolic acid.

6. A method according to claim 5, characterized in that the ester of the phenolic acid is octyl gallate or a lauryl gallate.

7. The method according to claim 1, characterized in that the pure substance or the plant extract or the mixture thereof is added into the circulation waters of the paper or board machine to a product concentration of 1 to 1000 ppm, preferably 10 to 100 ppm, calculated from the dry weight of the pure substance or plant extract.

8. The method according to claim 1, characterized in that the pure substance or the plant extract or the mixture thereof is applied into the circulation waters of the paper or board machine either periodically, preferably 2 to 8 times a day, or as a single dose once a day.

9. The method according to claim 1, characterized in that the pure substance or the plant extract of the mixture thereof is applied as a single dose into the containers containing adhering microbes, the dose being preferably 500 to 5000 ppm, as calculated from the dry weight of the pure substance or plant extract.

10. The method according to claim 1, characterized in that the thermophilic biofilm comprises at least one of the following adhering bacteria: Deinococcus geothermalis, Meiothermus silvanus, Burkholderia cepacia or Thermomonas sp., and/or the adhering mould Aspergillus fumigatus.

11. The use of a pure substance isolated from a plant or a plant extract or a mixture thereof for inhibiting the biofilm formation by thermophilic adhering microbes of paper and board machines on the surfaces of paper or board machines and/or for removing such biofilms from said surfaces.

12. The use according to claim 11, characterized in that the pure substance or plant extract or the mixture thereof is added to the circulation waters of the paper or board machine so as to obtain a product concentration of 1 to 1000 ppm, preferably 10 to 100 ppm, as calculated from the dry weight of the pure substance or the plant extract.

13. A method for determining the need of the addition of an anti-biofilm agent in paper or board manufacturing processes for the use in the method according to claim 1, characterized in that the method of determination comprises the following steps:
i) taking a sample from surfaces, a process water or raw materials of a paper or board machine which is to be monitored,
ii) if needed, removing any loose inorganic and/or organic material from the sample and suspending the remaining sample in an aqueous solution,
iii) shaking the suspended sample in a culturing device with a nutrient solution and, optionally, with an anti-biofilm agent,
iv) removing the growth solution together and with any material which may loose with said solution, such as any planktonic material and biofilm bacteria that are not adequately adhered, and staining the microbes adhered to the wall of the device, and
v) detecting qualitatively and/or quantitatively, on the basis of colour formation and intensity, the presence of adhering microbes in said suspended sample and, optionally, in said sample treated with said anti-biofilm agent.

14. The method according to claim 13, characterized in that the determination for monitoring the biofilm-forming microbes in a process is carried out before and/or after the plant-based anti-biofilm agent is added according to claims 1 to 10 into the process.

15. The method according to claim 13 or 14, characterized in that the determination is carried out for selecting an anti-biofilm plant extract or a pure substance isolated from a plant or a mixture thereof, which is suitable for the process in question, and for determining the effective concentration thereof.

16. The method according to claim 13, characterized in that at step (iii), the sample is shaken at a temperature of 35 to 65° C., preferably at 40 to 60° C., for 8 to 40 to 60° C., for 8 to 48 h, preferably for 12 to 24 h.

17. The method according to claim 13, characterized in that (i) the sample is taken from an deposit of slime or biofilm, (ii) the sample is suspended in a nutrient solution, and (iii) the suspended sample is applied into a recess of a culturing device, preferably into the wells of a well plate, in an amount of at least 1.5 ml, preferably about 2 to 5 ml, into each recess.

18. The method according to claim 13, characterized in that at stage (ii), the sample is first washed by mixing it with an aqueous solution, by settling the obtained mixture, and by removing the liquid phase above the settled sediment and, when needed, by repeating the procedure 5 to 10 times in total, after which the remaining sample is suspended in a nutrient solution, and (iii) the suspended sample thus obtained is applied into the wells of the well-plate.

19. The method according to claim 13, characterized in that (iii) the wells of the well-plate are filled with the suspended sample alone (=0 sample), the suspended sample together with one or more anti-biofilm agents in one or more concentrations, each anti-biofilm agent and/or concentration in each well, whereby at least one of the anti-biofilm agents is a pure substance or a plant extract isolated from a plant or a mixture thereof; and optionally a nutrient solution.

20-21. (canceled)
